(19) United States
(12) Patent Application Publication
(10) Pub. No.: US 2022/0242986 A1
(43) Pub. Date: Aug. 4, 2022

(54) SHAPE MEMORY RESIN, ARTICLE FOR EXTERNAL USE ON SKIN, AND METHOD FOR PRODUCING ARTICLE FOR EXTERNAL USE ON SKIN

(71) Applicant: Shiseido Company, Ltd., Tokyo, (JP)

(72) Inventors: Atsushi SOGABE, Tokyo (JP); Chunming WEN, Tokyo (JP); Jin GONG, Yamagata (JP)

(73) Assignee: Shiseido Company, Ltd., Tokyo (JP)

(21) Appl. No.: 17/618,691

(22) PCT Filed: May 27, 2020
(86) PCT No.: PCT/JP2020/020898

§ 371 Date: Dec. 13, 2021

(30) Foreign Application Priority Data
Jun. 14, 2019 (JP) 2019-111534

(51) Int. Cl. C08F 220/56; A45D 44/22; A61K 8/02; A61K 8/81

(52) U.S. Cl. CPC C08F 220/56; A45D 44/22; A61K 2800/54; A61K 8/8158; A61K 8/0212

(57) ABSTRACT

A shape memory resin includes a polymer comprising 64-85 mol % N-alkylacrylamide component, 14-35 mol % acrylic ester component, and 0.01-6 mol % crosslinking component.

### RELATED APPLICATION

The present application is based upon and claims the benefit of priority from Japanese Patent Application No. 2019-111534 filed on Jun. 14, 2019, the entire contents of which are incorporated herein by reference.

### TECHNICAL FIELD

The present disclosure relates to a resin having shape memory properties. The present disclosure relates to an article for external use on the skin (i.e., external skin article) including the resin. The present disclosure relates to a method for producing the external skin article.

### BACKGROUND ART

Face masks that employ shape memory resins are known in the art (e.g., Patent Literatures 1 and 2).

Patent Literature 1 discloses a face mask including a film obtained by molding a shape memory resin. Patent Literature 1 also discloses layering a cloth impregnated with a toner etc. onto one surface of the face mask.

Patent Literature 2 discloses a face mask sheet which is formed so as to cover the face by adhering tightly to the skin, wherein a shape memory material that contracts in response to body temperature is provided at least in a section corresponding to a sagging area of the skin, and the shape memory material is made to contract along the skin's sagging direction. Patent Literature 2 also discloses that the shape memory material is layered on a water-containable sheet material, and the water-containable sheet material is impregnated with a cosmetic solution.

### CITATION LIST

### Patent Literature

Patent Literature 1: Japanese Unexamined Patent Publication No. 2005-160833A

Patent Literature 2: Japanese Unexamined Patent Publication No. 2007-143586A

### SUMMARY OF INVENTION

### Technical Problem

The face masks disclosed in Patent Literatures 1 and 2 require a water-containable material for retaining cosmetic solutions etc. It is desirable, however, to be able to choose whether to use a water-containable material depending on the purpose and use, without necessarily requiring a water-containable material.

In the face masks disclosed in Patent Literatures 1 and 2, a water-containable material is layered on a shape memory resin. Unfortunately, in such a layered structure, the shape memory resin and the water-containable material cannot deform integrally. For example, at the time of contraction of the shape memory resin, it is difficult for the water-containable material to contract along with the shape memory resin. In such cases, the water-containable material may get twisted, for example.

Further, neither Patent Literature 1 nor Patent Literature 2 discloses a concrete composition of a shape memory material applicable to a face mask.

Hence, there is a demand for a shape memory resin that is applicable to the skin, and particularly, for a shape memory resin that is applicable integrally with a water-containable material. There is also a demand for an external skin article employing such a shape memory resin for cosmetic and/or beauty purposes, a method for using the same, and a method for producing the same.

### Solution to Problem

According to a first aspect of the present disclosure, a shape memory resin is provide, the shape memory resin comprising a polymer comprising from 64 to 85 mol % of a N-alkylacrylamide component, from 14 to 35 mol % of an acrylic ester component having a structure represented by Chem. 1 below, and from 0.01 to 6 mol % of a cross-linking component.

In the chemical formula shown in Chem. 1, R¹ is hydrogen or a C₁₋₄ alkyl group, alkenyl group or acyl group. R² is a C₁₀₋₂₀ alkyl group, alkenyl group or acyl group.

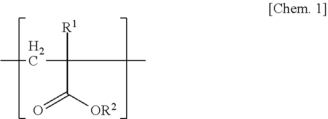

According to a second aspect of the present disclosure, an article for external use on the skin is provided, the article comprising the shape memory resin according to the first aspect.

According to a third aspect of the present disclosure, a method for producing an article for external use on the skin is provided, the production method comprising preparing a mixture by mixing a monomer including from 64 to 85 mol % of N-alkylacrylamide, from 14 to 35 mol % of an acrylic ester having a structure represented by Chem. 2 below and from 0.01 to 6 mol % of a cross-linking agent, and a polymerization initiator; producing a polymer by polymerizing the mixture; expanding the polymer by heating the polymer to a temperature equal to or above a glass transition temperature; and cooling the polymer in an expanded state.

In the chemical formula shown in Chem. 2, R³ is hydrogen or a C₁₋₄ alkyl group, alkenyl group or acyl group. R⁴ is a C₁₀₋₂₀ alkyl group, alkenyl group or acyl group.

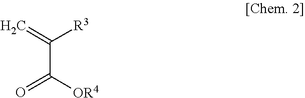

### Advantageous Effects of Invention

The shape memory resin of the present disclosure has temperature-responsive (temperature-dependent) shape memory properties. For example, the shape memory resin of the present disclosure can be induced to deform back to its original shape by human body temperature.

The shape memory resin of the present disclosure has high strength. Thus, the shape memory resin of the present disclosure can be formed thin. For example, the present shape memory resin can be formed in a film shape.

The shape memory resin of the present disclosure has high sustained stability in a shape memory state.

The shape memory resin of the present disclosure has biocompatibility. Thus, the shape memory resin of the present disclosure is applicable to the skin.

The shape memory resin of the present disclosure may have high transparency. Thus, the shape memory resin of the present disclosure may be applicable for many uses. For example, the shape memory resin can be made inconspicuous even when applied to the human body.

The external skin article of the present disclosure is applicable for stretching/contracting the skin.

According to the method for producing an external skin article of the present disclosure, it is possible to produce an external skin article according to the second aspect.

### DESCRIPTION OF EMBODIMENTS

Preferred modes of the aforementioned aspects will be described below.

According to a prefered mode of the above first aspect, the N-alkylacrylamide component comprises a N,N-dimethylacrylamide component.

According to a prefered mode of the above first aspect, the acrylic ester component comprises at least one selected from the group consisting of a stearyl acrylate component, a lauryl acrylate component, a 2-ethylhexyl acrylate component, and a butyl acrylate component.

According to a prefered mode of the above first aspect, the acrylic ester component comprises from 40 to 100 mol % of a stearyl acrylate component and from 0 to 60 mol % of a lauryl acrylate component with respect to a total amount of the acrylic ester component.

According to a prefered mode of the above first aspect, the cross-linking component comprises a N,N′-methylenebisacrylamide component.

According to a prefered mode of the above first aspect, the polymer has a Young's modulus of 1 MPa or less.

According to a prefered mode of the above first aspect, the polymer has an average strain of 500% or greater.

According to a prefered mode of the above first aspect, the shape memory resin further comprises polyethylene glycol.

According to a prefered mode of the above first aspect, a content by percentage of the polyethylene glycol is from 0.1 to 1% by mass with respect to the mass of the shape memory resin.

According to a prefered mode of the above first aspect, a water content of the shape memory resin is from 2 to 40% by mass with respect to the mass of the shape memory resin.

According to a prefered mode of the above first aspect, the shape memory resin has a temperature-responsive shape memory property.

According to a prefered mode of the above first aspect, the shape memory resin is flexible.

According to a prefered mode of the above second aspect, the article for external use on the skin further comprises a support. The support is impregnated with and/or covered by the shape memory resin.

According to a prefered mode of the above second aspect, the support is stretchable/contractile.

According to a prefered mode of the above second aspect, the support is at least one selected from the group consisting of nonwoven fabrics, woven fabrics, knitted fabrics, tufted fabrics, felts, fulled fabrics, paper, and water-containable materials.

According to a prefered mode of the above second aspect, the article has a sheet shape.

According to a prefered mode of the above second aspect, the article has a shape conforming to a surface shape of the skin.

According to a prefered mode of the above second aspect, the article has a shape conforming to at least a portion of the face.

According to a prefered mode of the above second aspect, the article is at least one selected from the group consisting of beauty products, cosmetic products, and therapeutic products.

According to a prefered mode of the above second aspect, the article further comprises a preparation for external application to the skin.

According to a prefered mode of the above third aspect, the production method further comprises placing the mixture in a mold before producing the polymer.

According to a prefered mode of the above third aspect, the production method further comprises placing a support in the mold. In the mold, the polymer is produced in a state where the support is impregnated with and/or covered by the mixture.

According to a prefered mode of the above third aspect, the support is stretchable/contractile.

According to a prefered mode of the above third aspect, the support is at least one selected from the group consisting of nonwoven fabrics, woven fabrics, knitted fabrics, tufted fabrics, felts, fulled fabrics, paper, and water-containable materials.

According to a prefered mode of the above third aspect, the mold has a shape of a portion of the face.

According to a prefered mode of the above third aspect, the production method comprises interposing a release sheet between the mixture and the mold.

According to a prefered mode of the above third aspect, the release sheet is a polytetrafluoroethylene sheet.

According to a prefered mode of the above third aspect, the article for external use on the skin is formed in a sheet shape.

According to a prefered mode of the above third aspect, the acrylic ester comprises at least one selected from the group consisting of stearyl acrylate, lauryl acrylate, 2-ethylhexyl acrylate, and butyl acrylate.

According to a prefered mode of the above third aspect, the acrylic ester contains from 40 to 100 mol % of stearyl acrylate and from 0 to 60 mol % of lauryl acrylate with respect to the total amount of the acrylic ester.

According to a prefered mode of the above third aspect, the N-alkylacrylamide comprises N,N-dimethylacrylamide.

According to a prefered mode of the above third aspect, the cross-linking agent comprises N,N′-methylenebisacrylamide.

A shape memory resin according to a first embodiment of the present disclosure will be described. In the following description, the polymer is described in terms of a monomer unit.

The shape memory resin of the present disclosure includes a polymer including a N-alkylacrylamide component, an acrylic ester component, and a cross-linking component.

### N-Alkylacrylamide Component

An example of the N-alkylacrylamide component may include a N,N-dimethylacrylamide component. The content of the N,N-dimethylacrylamide component may be 90 mol % or greater, 95 mol % or greater, or 100 mol %, with respect to the total amount of the N-alkylacrylamide component.

The content of the N-alkylacrylamide component may preferably be 64 mol % or greater, more preferably 70 mol % or greater, with respect to the total amount of components in the polymer. If the content of the N-alkylacrylamide component is less than 64 mol %, shape memory properties may deteriorate. The content of the N-alkylacrylamide component may preferably be 85 mol % or less, more preferably 80 mol % or less, with respect to the total amount of components in the polymer. If the content of the N-alkylacrylamide component exceeds 85 mol %, shape memory properties may deteriorate.

### Acrylic Ester Component

The acrylic ester component may have a structure represented by Chem. 1. In the chemical formula shown in Chem. 1, R¹ is hydrogen or a C₁₋₄ alkyl group, alkenyl group or acyl group, and R² is a C₁₀₋₂₀ alkyl group, alkenyl group or acyl group.

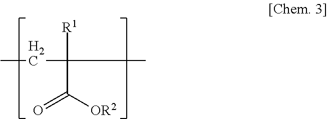

Examples of the acrylic ester component may include at least one of a stearyl acrylate component, a lauryl acrylate component, a 2-ethylhexyl acrylate component, or a butyl acrylate component. The total amount of the stearyl acrylate component, the lauryl acrylate component, the 2-ethylhexyl acrylate component, and the butyl acrylate component may be 90 mol % or greater, 95 mol % or greater, or 100 mol %, with respect to the total amount of the acrylic ester component.

For example, the acrylic ester component may include a stearyl acrylate component and a lauryl acrylate component. In this case, the content of the stearyl acrylate component may be, for example, 40 mol % or greater, 50 mol % or greater, 60 mol % or greater, or 65 mol % or greater, with respect to the total amount of the acrylic ester component. The content of the stearyl acrylate component may be, for example, 100 mol or less, 95 mol or less, 85 mol % or less, or 75 mol % or less, with respect to the total amount of the acrylic ester component. The content of the lauryl acrylate component may be, for example, 5 mol % or greater, 15 mol % or greater, or 25 mol % or greater, with respect to the total amount of the acrylic ester component. The content of the lauryl acrylate component may be, for example, 60 mol or less, 50 mol % or less, or 40 mol % or less, with respect to the total amount of the acrylic ester component. The stearyl acrylate component has an action of raising the glass transition temperature (Tg). Lauryl acrylate has an action of lowering the glass transition temperature. By mixing the stearyl acrylate component and the lauryl acrylate component, it is possible to adjust the contraction temperature at which shape recovery occurs.

In cases where the acrylic ester component includes a stearyl acrylate component and a lauryl acrylate component, the molar ratio between the stearyl acrylate component and the lauryl acrylate component may be 1 mol or greater, 1.5 mol or greater, or 2 mol or greater, of the stearyl acrylate component with respect to 1 mol of the lauryl acrylate component. The molar ratio may be 4 mol or less, 3.5 mol or less, or 3 mol or less, of the stearyl acrylate component with respect to 1 mol of the lauryl acrylate component.

The content of the acrylic ester component may preferably be 14 mol % or greater, more preferably 20 mol % or greater, with respect to the total amount of components in the polymer. If the content of the acrylic ester component is less than 14 mol %, shape memory properties may deteriorate. The content of the acrylic ester component may preferably be 35 mol % or less, more preferably 30 mol % or less, with respect to the total amount of components in the polymer. If the content of the acrylic ester component exceeds 35 mol %, shape memory properties may deteriorate.

### Cross-Linking Component

An example of the cross-linking component may include a N,N′-methylenebisacrylamide component.

The content of the cross-linking component may be 0.01 mol % or greater, 0.03 mol % or greater, 0.05 mol % or greater, 0.1 mol % or greater, 0.3 mol % or greater, 0.7 mol % or greater, 1 mol % or greater, 1.5 mol % or greater, 2 mol % or greater, 2.5 mol % or greater, 3 mol % or greater, 3.5 mol % or greater, or 4 mol % or greater, with respect to the total amount of components in the polymer. The content of the cross-linking component may be 6 mol % or less, 5 mol % or less, 4 mol % or less, 3 mol % or less, 2 mol % or less, 1 mol % or less, 0.8 mol % or less, 0.5 mol % or less, 0.2 mol % or less, 0.1 mol % or less, or 0.08 mol % or less, with respect to the total amount of components in the polymer. By adjusting the content by percentage of the cross-linking component, it is possible to adjust the average strain, Young's modulus, and tensile strength. Further, by reducing the content of the cross-linking component, it is possible to improve sustained stability of the shape memory state at room temperature.

### Polymerization Initiator

The shape memory resin of the present disclosure may further include a polymerization initiator. Examples of the polymerization initiator may include at least one of benzophenone, a 2,2′-azobis(isobutyronitrile) component, a 2,2′-azobis(2-methylpropionamidine) dihydrochloride component, a 4,4′-azobis(4-cyanovaleric acid) component, or a dimethyl 2,2′-azobis(isobutyrate) component.

The content of the polymerization initiator may be 0.02 mol % or greater, or 0.05 mol % or greater, with respect to the total amount of components in the shape memory resin. The content of the polymerization initiator may be 0.2 mol % or less, or 0.15 mol % or less, with respect to the total amount of components in the shape memory resin.

### Water Containability

The shape memory resin of the present disclosure may further include water. The content by percentage of water may be, for example, 2% by mass or greater, 3% by mass or greater, 5% by mass or greater, 10% by mass or greater, 20% by mass or greater, or 30% by mass or greater, with respect to the mass of the shape memory resin. The content by percentage of water may be, for example, 40% by mass or less, 30% by mass or less, 20% by mass or less, 10% by mass or less, 8% by mass or less, or 7% by mass or less, with respect to the mass of the shape memory resin.

### Others

The shape memory resin of the present disclosure may contain, as appropriate, various additives such as preparations for external application to the skin (external skin preparations), antioxidants, thermostabilizers, slip additives, antistatic agents, plasticizers, pigments, etc., depending on the use and purpose of molding. These additive components may be blended in either the polymerization reaction step or the processing/molding step. In cases where the shape memory resin is to be applied to the skin, the shape memory resin may contain, for example, at least one of moisturizers, film-forming agents, UV absorbers, amino acids, pH adjusters, skin nutrients, vitamins, perfumes, etc., as external skin preparations. The shape memory resin of the present disclosure may contain an external skin preparation in the form of an aqueous solution.

The shape memory resin of the present disclosure may contain an alcohol. Examples of the alcohol may include lower alcohols, polyols, etc. An example of a polyol may include polyethylene glycol. The content by percentage of polyethylene glycol may be, for example, 0.001 mol % or greater, or 0.002 mol % or greater, with respect to the total amount of monomer(s) in the shape memory resin. The content by percentage of polyethylene glycol may be, for example, 0.004 mol % or less, or 0.003 mol % or less, with respect to the total amount of monomer(s) in the shape memory resin.

The shape memory resin of the present disclosure may have a sheet shape, for example. The shape memory resin of the present disclosure may have a thickness appropriate for the use and purpose. The shape memory resin of the present disclosure may have a thickness of, for example, 0.8 mm or greater, 1 mm or greater, 1.5 mm or greater, 2 mm or greater, 2.5 mm or greater, or 3 mm or greater. The shape memory resin of the present disclosure may have a thickness of, for example, 4 mm or less, 3.5 mm or less, 3 mm or less, 2.5 mm or less, 2 mm or less, or 1.5 mm or less. The shape memory resin of the present disclosure may have a shape conforming to the surface of the skin, e.g., a shape conforming to at least a portion of the face.

### Physical Properties and Characteristics

The shape memory resin of the present disclosure may have the following physical properties and/or characteristics.

The shape memory resin of the present disclosure may be transparent or semi-transparent (translucent).

The shape memory resin of the present disclosure may have an average strain at break of 50% or greater, 80% or greater, 100% or greater, 150% or greater, 200% or greater, 300% or greater, 400% or greater, 500% or greater, or 600 or greater. The shape memory resin of the present disclosure may have an average strain at break of 1000% or less, 900% or less, 800% or less, 700% or less, 600% or less, 500% or less, 400% or less, 300% or less, 200% or less, or 150% or less. The average strain at break can be measured according to JIS K6251. The average strain can be adjusted by adjusting the added amount of cross-linking agent. By increasing the average strain, it is possible to improve the contraction effect caused by shape recovery.

The shape memory resin of the present disclosure may have a Young's modulus of 0.01 MPa or greater, 0.1 MPa or greater, 1 MPa or greater, 2 MPa or greater, 5 MPa or greater, or 10 MPa or greater. The shape memory resin of the present disclosure may have a Young's modulus of 20 MPa or less, 10 MPa or less, 5 MPa or less, 1 MPa or less, 0.5 MPa or less, or 0.1 MPa or less. The Young's modulus can be measured according to JIS K6251. The Young's modulus can be adjusted by adjusting the added amount of cross-linking agent. By increasing the Young's modulus, it is possible to increase the contraction force upon shape recovery.

The shape memory resin of the present disclosure may have a tensile strength at break of 0.5 MPa or greater, 1 MPa or greater, 2 MPa or greater, 3 MPa or greater, 5 MPa or greater, or 7 MPa or greater. The shape memory resin of the present disclosure may have a tensile strength at break of 15 MPa or less, 10 MPa or less, 8 MPa or less, 5 MPa or less, or 3 MPa or less. The tensile strength at break can be measured according to JIS K6251.

The shape memory resin of the present disclosure has an endothermic peak within 28° C. to 48° C.

### Shape Memory Properties

The shape memory properties of the shape memory resin of the present disclosure, as well as methods for using the shape memory resin of the present disclosure (methods for inducing the shape memory properties), will be described.

The shape memory resin of the present disclosure has shape memory properties. Particularly, the resin composition of the present disclosure has temperature-responsive (temperature-dependent) shape memory properties. For example, the shape memory resin of the present disclosure can be induced to deform back to its original shape by human body temperature.

First, a shape memory resin having a sheet shape is heated to a deformable temperature. For example, the shape memory resin is heated to a temperature within a range from the glass transition temperature to the melting point. The heating temperature may preferably be 50° C. or higher, for example. The heating temperature may preferably be 70° C. or lower, for example.

Next, the softened shape memory resin is stretched to such an extent that it does not break. For example, the shape memory resin may be stretched in one direction to 1.2 times or greater, 1.5 times or greater, 2 times or greater, 3 times or greater, or 5 times or greater, its original length. For example, the shape memory resin may preferably be stretched in one direction to an extent of 10 times or less its original length.

The stretching direction is not limited to a uniaxial direction. For example, the shape memory resin may be expanded in the planar direction (biaxial directions). In cases of expanding the shape memory resin in the planar direction, the resin may be expanded to 1.5 times or greater, 2 times or greater, 3 times or greater, or 5 times or greater, its original area. For example, the shape memory resin may preferably be expanded to an extent of 10 times or less its original area.

Next, the shape memory resin is cooled in the stretched or expanded state. For example, the shape memory resin may be cooled to 10° C. or lower, preferably 5° C. or lower, or 0° C. or lower. In this way, the shape memory resin is shaped in its stretched or expanded state and memorizes its original shape. Once the resin is fixed in the stretched or expanded state, the shape memory resin is returned to room temperature (preferably 25° C. or lower).

To induce the shape memory properties, the shape memory resin is heated/warmed. For example, the shape memory resin may preferably be heated/warmed to 30° C. or higher. The heating/warming temperature may preferably be 45° C. or lower, more preferably 40° C. or lower. For example, the shape memory resin may be heated/warmed by human body temperature. In this way, the shape memory resin contracts to its original shape. For example, the shape memory resin can contract in the stretched axial direction by 20% to 30% with respect to its length before contraction. The shape memory resin can contract by 20% to 60% with respect to its area before contraction.

The shape memory resin of the present disclosure may be a shape memory resin that exhibits the following shape memory properties. The shape memory resin may have the aforementioned composition and/or may be produced according to production methods described below.

First, a 20-mm-long, 10-mm-wide, 1-mm-thick sheet of shape memory resin is prepared (first step). After the first step, the sheet is heated to a temperature within 50° C. to 70° C., to soften the shape memory resin (second step). In a softened state achieved by the second step, the sheet is stretched in the length direction to 1.5 times its original length (third step). The sheet is cooled to 0° C. in a stretched state achieved by the third step (fourth step). After the fourth step, the sheet is left to stand at room temperature of 25° C. or lower (fifth step). After being left to stand, the sheet has a length of 26 mm or greater. After the fifth step, the sheet is heated to a temperature within 32° C. to 45° C. (sixth step). The sheet contracts in the sixth step. After contraction, the sheet may have a length of 24 mm or less.

The length of the shape memory resin after restoration (after contraction) may preferably be 130% or less, more preferably 120% or less, even more preferably 110% or less, with respect to the original length before stretching.

According to the shape memory resin of the present disclosure, the resin can be made to memorize a shape. Shape restoration can be induced by heating. Particularly, shape restoration can be induced by human body temperature. Thus, the shape memory resin of the present disclosure can be deformed by being applied to the human skin.

The shape memory resin of the present disclosure has excellent shape memory properties capable of retaining a shape memory state. Thus, the shape memory resin can be stored for a long time in a shape-memorizing state (i.e., stretched state before deformation).

The shape memory resin of the present disclosure has water containability. Thus, the shape memory resin of the present disclosure can retain a component for external application to the skin (i.e., external skin component) such as a beauty component.

The shape memory resin of the present disclosure is applicable, for example, to beauty products, cosmetic products, medical products (therapeutic products), clothing products, shoe products, etc. The shape memory resin of the present disclosure is also applicable, for example, to masks.

A method for producing the shape memory resin of the present disclosure will be described.

The shape memory resin of the present disclosure can be produced by: mixing a monomer including a N-alkylacrylamide, an acrylic ester and a cross-linking agent, and a polymerization initiator; and polymerizing the monomer.

An example of the N-alkylacrylamide may include N,N-dimethylacrylamide.

Examples of the acrylic ester may include compounds having a structure represented by Chem. 2 below. In the chemical formula shown in Chem. 2, R³ is hydrogen or a C₁₋₄ alkyl group, alkenyl group or acyl group, and R⁴ is a C₁₀₋₂₀ alkyl group, alkenyl group or acyl group.

For the acrylic ester, it is possible to use at least one of stearyl acrylate, lauryl acrylate, 2-ethylhexyl acrylate, or butyl acrylate. Particularly, the acrylic ester may be stearyl acrylate and/or lauryl acrylate.

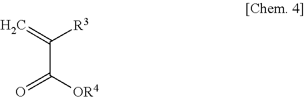

For the cross-linking agent, it is possible to use, for example, at least one of N,N′-methylenebisacrylamide, N,N′ -methylenebisacrylamide, 2,2′-azobis(isobutyronitrile), 2,2′-azobis(2-methylpropionamidine) dihydrochloride, 4,4′-azobis(4-cyanovaleric acid), or dimethyl 2,2′-azobis(isobutyrate).

For the polymerization initiator, it is possible to use, for example, at least one of benzophenone, a 2,2′-azobis(isobutyronitrile) component, a 2,2′-azobis(2-methylpropionamidine) dihydrochloride component, a 4,4′-azobis(4-cyanovaleric acid) component, or a dimethyl 2,2′-azobis(isobutyrate) component.

The content by percentage of the monomer and the polymerization initiator may be the same as the content by percentage of the respective components described in the first embodiment.

The shape memory resin of the present disclosure can be produced by polymerizing the aforementioned monomer by a known method.

The shape memory resin can be shaped into a sheet shape by a known method.

An article for external use on the skin (i.e., external skin article) according to a second embodiment of the present disclosure will be described.

The external skin article of the present disclosure includes the shape memory resin according to the first embodiment.

The external skin article of the present disclosure may further include a support for supporting the shape memory resin. The support may serve as a carrier for carrying the shape memory resin.

The support may have at least one of porosity, water containability, stretchability/contractility, or flexibility.

The support may be, for example, at least one of nonwoven fabrics, woven fabrics, knitted fabrics, tufted fabrics, felts, fulled fabrics, paper, or water-containable materials.

The support may have, for example, a sheet shape. The support may have, for example, a shape conforming to the skin surface. The support may have, for example, a shape conforming to at least a portion of the face.

The support may have a thickness of, for example, 0.5 μm or greater, 1 μm or greater, 5 μm or greater, 10 μm or greater, 20 μm or greater, 50 μm or greater, 100 μm or greater, 200 μm or greater, 500 μm or greater, 1 mm or greater, or 2 mm or greater. The support may have a thickness of, for example, 5 mm or less, 2 mm or less, 1 mm or less, 500 μm or less, 200 μm or less, 100 μm or less, 50 μm or less, or 10 μm or less.

At least a portion of the support may be impregnated with the shape memory resin. At least a portion of the support may be covered by or layered with the shape memory resin. The shape memory resin may cover, or be layered on, one surface of the support, or may cover, or be layered on, both surfaces of the support. Preferably, the support is deformable according to deformation of the shape memory resin.

The external skin article of the present disclosure may further include a component for external application to the skin (i.e., external skin component). The external skin component can be contained in the shape memory resin and/or the support. The external skin component may be an aqueous composition, an emulsion-type composition, or an oily composition containing the component exemplified below. The external skin component may be, for example, a cosmetic component and/or a beauty component. Examples of the external skin component may include moisturizers, skin nutrients, vitamins, antiphlogistic agents, skin whitening agents, various extracts, blood circulation promoting agents, anti-seborrheic agents, anti-inflammatory agents, pH adjusters, perfumes, therapeutic drugs, etc.

The external skin article of the present disclosure enables the shape memory resin according to the first embodiment to be applied to the skin more easily. Further, the external skin component can be applied to the skin more easily. Furthermore, the deformation of the shape memory resin can be conveyed to the skin more easily.

A method for producing the external skin article according to the second embodiment will be described. Below is an example of a method for producing an external skin article in which a support is impregnated with a shape memory resin.

First, a mold is prepared for shaping the external skin article. The mold may have, for example, a shape of a portion of the skin. The mold may have, for example, a shape of at least a portion of the face.

Next, the aforementioned monomer, a polymerization initiator, and a support are placed in the mold. The monomer and the polymerization initiator may be the same as described above. The support may be the same as described above.

A release sheet for allowing the external skin article to be removed from the mold easily may be interposed between the mold and the monomer and/or support. For the release sheet, it is possible to use, for example, a polytetrafluoroethylene sheet.

Next, the monomer is polymerized, to produce an external skin article having the shape of the mold and in which the support is impregnated with and/or layered with the shape memory resin.

In the above example, the monomer was placed in a mold, but instead, an already-produced shape memory resin and a support may be placed in a mold and the shape memory resin may be heated to a temperature equal to or above the glass transition temperature or melting point, to impregnate/cover/layer the support with the shape memory resin.

Next, in cases of including an external skin component such as a beauty component etc., the external skin article may be impregnated with a liquid containing the external skin component.

Next, a method for using the external skin article will be described.

The method for using the external skin article is the same as the method for using the aforementioned shape memory resin. After making the shape memory resin memorize its original shape, the external skin article is attached to a predetermined portion of the skin. The shape memory resin can then be made to deform by body temperature, to apply deformation force to the skin.

The external skin article of the present disclosure is applicable, for example, to beauty products, cosmetic products, medical products (therapeutic products), clothing products, shoe products, etc. The external skin article of the present disclosure is also applicable, for example, to masks.

The shape memory resin and the external skin article of the present disclosure may encompass shape memory resins and external skin articles obtained by production methods described in the present disclosure. In some cases, features of the shape memory resin and the external skin article of the present disclosure, other than the features described above, may be difficult to directly define according to the composition, structure, characteristics, etc., of the shape memory resin and the external skin article of the present disclosure. In such cases, it should be permissible to define the shape memory resin and the external skin article of the present disclosure according to methods for producing the same.

### EXAMPLES

The shape memory resin of the present disclosure will be described below by way of examples. The shape memory resin of the present disclosure is, however, not limited to the following examples.

### Test Examples 1 to 7

Shape memory resins having different compositions were prepared, and the physical properties of each shape memory resin were measured. Table 1 shows the blending ratio of materials of the respective shape memory resins that were prepared. The blending ratio can be considered to be the same as the composition of each shape memory resin. The blending ratio of the monomer, the cross-linking agent, and the initiator shown in Table 1 is the respective ratio with respect to the total amount of the monomer, the cross-linking agent, and the initiator.

In each composition, the average strain at break, Young's modulus, tensile strength at break, sustained stability, and shape memory properties were measured. The average strain at break, Young's modulus, and tensile strength at break were measured according to JIS K6251, wherein both ends of a size-7 dumbbell-shaped test piece were pulled with a tensile tester at a rate of 100 mm/minute. The average strain and tensile strength at break are values at the point of rupture.

Stretchability was found by measuring the stretch ratio by subjecting a JIS-K6251-compliant size-7 dumbbell-shaped test piece to a single-direction load at room temperature, and evaluating the same according to the following criteria.

A: The stretching degree was 200% or greater with respect to the original length.

B: The stretching degree was less than 200% with respect to the original length.

Sustained stability (Temporal stability) was found as follows. First, a 20-mm-long, 10-mm-wide, 1-mm-thick test piece was heated to 50° C. to soften, and then the test piece was stretched in a uniaxial direction (length direction) to a length twice (200%) the original length. Next, the test piece, in its stretched state, was cooled to 0° C., to thereby fix the stretched state. The stretched-state test piece was left to stand in a 5° C. constant-temperature oven for 24 hours, and the extent to which the test piece contracted was evaluated according to the following criteria. Sustained stability at 25° C. was tested in the same manner. Sustained stability was tested for the resins of Test Examples 3 and 4. For the resin of Test Example 3, sustained stability was tested in the same manner also with a 2-mm-thick test piece.

A: The length after being left to stand for 24 hours was 190% or greater with respect to the original length.

B: The length after being left to stand for 24 hours was 160% or greater to less than 190% with respect to the original length.

C: The length after being left to stand for 24 hours was 130% or greater to less than 160% with respect to the original length.

Shape memory properties were found as follows. First, a 20-mm-long, 10-mm-wide, 1-mm-thick test piece was heated to 50° C. to soften, and then the test piece was stretched in a uniaxial direction (length direction) to a length 1.5 times (150%) the original length. Next, the test piece, in its stretched state, was cooled to 0° C., to thereby fix the stretched state. Next, the stretched-state test piece was heated to 40° C. and caused to contract. The extent to which the test piece contracted with respect to the original length (i.e., recovered its shape) was evaluated according to the following criteria.

A: The length after shape recovery was greater than 100% to 110% or less of the original length.

B: The length after shape recovery was greater than 110% to 120% or less of the original length.

C: The length after shape recovery was greater than 120% of the original length.

The measurement results are shown in Table 2. The average strain, Young's modulus, and tensile strength are each an average value found by performing measurement five times.

“Water content after synthesis” as shown in Table 1 indicates the water content in a state where the shape memory resin, after synthesis, is left to stand at atmospheric temperature. “Saturated water content” indicates the water content in a saturated state (equilibrium swollen state) wherein the weight of the shape memory resin has become invariant by immersing the shape memory resin in pure water at atmospheric temperature for at least two days. The water content was measured with an electronic moisture meter (MOC-120H from Shimadzu Corporation). The water content was not measured in Test Examples 4 to 6.

It was possible to produce a shape memory resin from N,N-dimethylacrylamide and stearyl acrylate. It was also found that a shape memory resin could be prepared by further adding lauryl acrylate. It was verified that all of the resins of Test Examples 1 to 6 had sufficient shape memory properties near human body temperature.

A tendency was found that adding large amounts of cross-linking agent reduces average strain but increases Young's modulus. The physical properties of Table 2 show that all of the shape memory resins of Test Examples 1 to 6, when attached to the skin and caused to undergo shape recovery, were capable of exerting a significant force to the skin by deforming.

Compared to other Test Examples, it was possible to improve the stretchability in Test Examples 3 and 4, which included a N,N-dimethylacrylamide component, a stearyl acrylate component, and a lauryl acrylate component as constituent units and also included from 0.01 to 1 mol % of a cross-linking agent. It should be noted that, even Test Examples 1, 2, 5 and 6, which were rated “B” in terms of stretchability, were capable of being stretched up to at least 1.5 times its original length and thus had sufficient stretchability to be used as a shape memory resin as described above.

It was verified that, when the shape memory resin was stored at a low temperature (5° C.) in a shape memory state (stretched state), the resin hardly contracted. A certain degree of contraction was observed when the resin was stored at a temperature closer to body temperature, but the shape memory resin was in a sufficiently usable state as a shape memory resin. It was thus verified that the shape memory resin of the present disclosure had sustained (temporal) stability in a shape memory state (stretched state). It was also verified that test pieces with thicknesses of 1 mm and 2 mm both had the same level of sustained stability. It is thought that, to improve sustained stability, it is preferred to reduce the added amount of the cross-linking agent to, for example, 0.2 mol % or less, preferably 0.1 mol % or less.

The water content was substantially constant, regardless of composition. After synthesis, each shape memory resin had a water content of from 4 to 7% by mass. The water content in a saturated state was from 30 to 35% by mass. It is thought that the water content is adjustable by adjusting the time of immersion in water. Since the shape memory resin has water containability, the shape memory resin itself can retain, for example, an aqueous solution of an external skin component.

The shape memory resins of Test Examples 1 to 6 were all semi-transparent (translucent).

TABLE 1

| | | | | Cross- | | | | |
|---|---|---|---|---|---|---|---|---|
| | | | | linking | | | Water content | |
| | | Monomer | | agent | Initiator | Polyol | After | |
| Test | DMAA^{*1} | SA^{*2} | DA^{*3} | MBAA^{*4} | BP^{*5} | PEG^{*6} | synthesis | Saturated |
| Example | mol % | mol % | mol % | mol % | mol % | mol % | wt % | wt % |
| 1 | 75 | 25 | 0 | 0.05 | 0.1 | 0 | 5.4 | 34 |
| 2 | 75 | 25 | 0 | 0.05 | 0.1 | 0.0025 | 6.3 | 31 |
| 3 | 75 | 17.5 | 7.5 | 0.05 | 0.1 | 0 | 4.6 | 35 |
| 4 | 75 | 17.5 | 7.5 | 0.5 | 0.1 | 0 | — | — |
| 5 | 75 | 17.5 | 7.5 | 1.5 | 0.1 | 0 | — | — |
| 6 | 75 | 17.5 | 7.5 | 4.75 | 0.1 | 0 | — | — |

^{*1}N,N-dimethylacrylamide
^{*2}Stearyl acrylate (octadecyl acrylate)
^{*3}Lauryl acrylate (dodecyl acrylate)
^{*4}N,N’-methylenebisacrylamide
^{*5}Benzophenone
^{*6}PEG400

TABLE 2

| | Average | Young’s | Tensile | | | | |
|---|---|---|---|---|---|---|---|
| Test | strain | modulus | strength | Stretch- | Sustained stability | | Shape memory |
| Example | (%) | (MPa) | (MPa) | ability | 5° C. | 25° C. | properties |
| 1 | 274 | 14.1 | 6.7 | B | — | — | A |
| 2 | 284 | 6.6 | 6.6 | B | — | — | A |
| 3 | 840 | 0.0026 | 9.2 | A | A | B | A |
| 4 | 592 | 0.29 | 1.7 | A | A | C | A |
| 5 | 101 | 4.91 | 4.9 | B | — | — | A |
| 6 | 101 | 4.83 | 4.9 | B | — | — | A |

The shape memory resin, the article for external use on the skin (i.e., external skin article), and the methods for producing thereof according to the present invention have been described according to the foregoing embodiments and examples, but the invention is not limited to the foregoing embodiments and examples and may encompass various transformations, modifications, and improvements made to the various disclosed elements (including elements disclosed in the Claims, Description, and Drawings) within the scope of the invention and according to the fundamental technical idea of the present invention. Further, various combinations, substitutions, and selections of the various disclosed elements are possible within the scope of the claims of the invention.

Further issues, objectives, and embodiments (including modifications) of the present invention are revealed also from the entire disclosure of the invention including the Claims.

The numerical ranges disclosed herein are to be construed in such a manner that arbitrary numerical values and ranges falling within the disclosed ranges are treated as being concretely described herein, even where not specifically stated.

Some or all of the foregoing embodiments may be described as in the following additional items, although not limited thereto. The various additional items may be employed in combination with the claim(s) in the Scope of Claims.

### Additional Item 1

A shape memory resin, comprising:

a sheet shape, wherein, when the resin is heated from a temperature of 30° C. or lower to a temperature within 32° C. to 45° C., the length thereof contracts to 80% or less of the length before heating.

### Additional Item 2

The shape memory resin, wherein, when the shape memory resin sheet having a flat shape is heated to a temperature within 32° C. to 45° C., the sheet deforms so as to have the shape of a portion of the skin.

### Additional Item 3

The shape memory resin, wherein:

when undergoing
a first step of preparing a 20-mm-long, 10-mm-wide, 1-mm-thick sheet of the shape memory resin,
a second step of heating the sheet to a temperature within 50° C. to 70° C. after the first step,
a third step of stretching, in a softened state achieved by the second step, the sheet in the length direction to 1.5 times its original length,
a fourth step of cooling the sheet to 0° C. in a stretched state achieved by the third step, and
a fifth step of leaving the sheet to stand at a room temperature of 25° C. or lower after the fourth step,

the sheet has a length of 26 mm or greater; and

the sheet contracts when undergoing a sixth step of heating the sheet to a temperature within 32° C. to 45° C. after the fifth step.

### Additional Item 4

The shape memory resin as described in the Additional Item, wherein, after the sixth step, the sheet has a length of 24 mm or less.

### Additional Item 5

The shape memory resin as described in the Additional Item, wherein, after preparation of the shape memory resin, the water content of the shape memory resin in a state left to stand in air at room temperature is from 2 to 8% by mass with respect to the mass of the shape memory resin.

### Additional Item 6

The shape memory resin as described in the Additional Item, wherein the saturated water content of the shape memory resin is from 25 to 40% by mass with respect to the mass of the shape memory resin.

### Additional Item 7

The shape memory resin as described in the Additional Item, wherein the resin has an endothermic peak within 28° C. to 48° C.

### Additional Item 8

The shape memory resin as described in the Additional Item, further containing an aqueous solution containing a preparation for external application to the skin (external skin preparation).

### Additional Item 9

The shape memory resin as described in the Additional Item, wherein the resin has a sheet shape that is from 0.01 to 3 mm thick.

### Additional Item 10

The shape memory resin as described in the Additional Item, wherein the resin has a shape conforming to a surface shape of the skin.

### Additional Item 11

The shape memory resin as described in the Additional Item, wherein the resin has a shape conforming to a portion of the face.

### Additional Item 12

A cosmetic method and/or a beauty method, involving a step of attaching, to the skin, the shape memory resin according to the first embodiment and/or the external skin article according to the second embodiment.

### Additional Item 13

The cosmetic method and/or beauty method as described in the Additional Item, wherein the shape memory resin and/or the external skin article are/is attached to the face.

### Additional Item 14

The cosmetic method and/or beauty method as described in the Additional Item, wherein, after being attached to the skin, the shape memory resin and/or the external skin article contract(s) on the skin by body temperature.

### INDUSTRIAL APPLICABILITY

The shape memory resin, the article for external use on the skin (i.e., external skin article), and the method for producing the external skin article according to the present disclosure are applicable to cosmetic products, beauty products, and therapeutic products.

1. A shape memory resin comprising:
a polymer comprising:
from 64 to 85 mol % of a N-alkylacrylamide component;
from 14 to 35 mol % of an acrylic ester component having a structure represented by Chem. 1 below; and
from 0.01 to 6 mol % of a cross-linking component and polyethylene glycol: 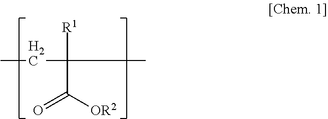
(in the chemical formula shown in Chem. 1, R¹ is hydrogen or a C₁₋₄ alkyl group, alkenyl group or acyl group, and R² is a C₁₀₋₂₀ alkyl group, alkenyl group or acyl group).

2. The shape memory resin according to claim 1, wherein the N-alkylacrylamide component comprises a N,N-dimethylacrylamide component.

3. The shape memory resin according to claim 1, wherein the acrylic ester component comprises at least one selected from the group consisting of a stearyl acrylate component, a lauryl acrylate component, a 2-ethylhexyl acrylate component, and a butyl acrylate component.

4. The shape memory resin according to claim 1, wherein the acrylic ester component comprises from 40 to 100 mol % of a stearyl acrylate component and from 0 to 60 mol % of a lauryl acrylate component with respect to a total amount of the acrylic ester component.

5. The shape memory resin according to claim 1, wherein the cross-linking component comprises a N,N′-methylenebisacrylamide component.

6. The shape memory resin according to claim 1, wherein the polymer has a Young's modulus of 1 MPa or less.

7. The shape memory resin according to claim 1, wherein the polymer has an average strain of 500% or greater.

8. (canceled)

9. The shape memory resin according to claim 1, wherein a content by percentage of the polyethylene glycol is from 0.1 to 1% by mass with respect to the mass of the shape memory resin.

10. The shape memory resin according to claim 1, wherein a water content of the shape memory resin is from 2 to 40% by mass with respect to the mass of the shape memory resin.

11. The shape memory resin according to claim 1, wherein the shape memory resin has a temperature-responsive shape memory property.

12. The shape memory resin according to claim 1, wherein the shape memory resin is flexible.

13.-34. (canceled)

35. A method of applying an article for external use on the skin, comprising:
attaching the article comprising a shape memory resin to the skin;
wherein the shape memory resin comprises:
a polymer comprising:
from 64 to 85 mol % of a N-alkylacrylamide component;
from 14 to 35 mol % of an acrylic ester component having a structure represented by Chem. 2 below; and
from 0.01 to 6 mol % of a cross-linking component: 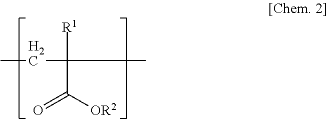
(in the chemical formula shown in Chem. 2, R¹ is hydrogen or a C₁₋₄ alkyl group, alkenyl group or acyl group, and R² is a C₁₀₋₂₀ alkyl group, alkenyl group or acyl group).

36. The method according to claim 35,
wherein the shape memory resin further comprises a support which is impregnated with and/or covered by the shape memory resin.

37. The method according to claim 36, wherein the support is stretchable/contractile.

38. The method according to claim 36, wherein the support is at least one selected from the group consisting of nonwoven fabrics, woven fabrics, knitted fabrics, tufted fabrics, felts, fulled fabrics, paper, and water-containable materials.

39. The method according to claim 35, wherein the article has a sheet shape.

40. The method according to claim 35, wherein the article has a shape conforming to a surface shape of the skin.

41. The method according to claim 35, wherein the article has a shape conforming to at least a portion of the face.

42. The method according to claim 35, wherein the method is at least one selected from the group consisting of beauty methods, cosmetic methods, and therapeutic methods.

43. The method according to claim 35, wherein the article further comprises a preparation for external application to the skin.
